(19) United States
(12) Patent Application Publication
(10) Pub. No.: US 2002/0183783 A1
(43) Pub. Date: Dec. 5, 2002

(54) Guidewire for capturing emboli in endovascular interventions

(76) Inventor: John H. Shadduck, Tiburon, CA (US)

Correspondence Address: John H. Shadduck, 1490 Vistazo West, Tiburon, CA, 94920, US

(21) Appl. No.: 10/160,801

(22) Filed: Jun. 1, 2002

Related U.S. Application Data
(60) Provisional application No. 60/295,939, filed on Jun. 4, 2001.

(51) Int. Cl. A61M 29/00

(52) U.S. Cl. 606/200

(57) ABSTRACT

A guidewire device and methods for containing and removing embolic materials from within a vascular system. The guidewire provides a very low profile expandable structure that can be carried at the distal end of any guidewire used in an endovascular intervention. The structure can be expanded at a location distal to a targeted treatment site, and due to its very low profile when non-expanded, can be passed through any narrow and tortuous occluded vessels that can accommodate a guidewire. The expandable structure comprises a thin film filter portion coupled to at least one support portion for supporting the filter portion in an expanded state. The support portion in its first non-extended state comprises at least one tensioned nitinol member constrained by an electrolytic sacrificial weld. The guidewire is coupled to a remote electrical source and controller for causing electrolysis of the sacrificial component of the invention.

### CROSS-REFERENCE TO RELATED APPLICATION

This application claims priority from Provisional U.S. Patent Application Ser. No. 60/295,939 filed Jun. 4, 2001 (Docket No. S-AES-001) having the same title as this disclosure, which is incorporated herein by reference.

### BACKGROUND OF THE INVENTION

1. Field of the Invention

The present invention relates to guidewire devices and methods for containing and removing embolic materials from within an endovascular treatment site. More in particular, the invention provides a system for maintaining a very low profile working end of a guidewire that can expand in cross-section to open a filter sac to capture embolic particles. The working end thus can be passed through any narrow or tortuous occluded vessels that can accommodate a guidewire of a standard dimension. Thereafter, the guidewire's working end can be expanded by means of a sacrificial coupling to deploy a filter sac distal to a targeted treatment site. The device is particularly suited for neurothrombectomy and embolectomy procedures, and a paired guidewire system with two identical low profile expandable working ends can be sued to provide distal protection in two branch arteries for thrombectomy at a branch location.

2. Description of the Related Art

Interventional cardiology procedures for treating occlusive vascular disease, such as angioplasty, thrombectomy, atherectomy or stent placement, can result in embolic material migrating downstream from the treatment site. Such embolic particles often are large and can occlude small vessels, for example, resulting in embolic stroke. Such ischemia can threaten the patient's life. Emboli also can lodge in the heart or lungs.

Various devices have been proposed for reducing the risk of emboli by blocking or capturing emboli with the downstream deployment of a balloon, filter, basket or similar structure. A particular disadvantage of all prior art systems is the large cross-section of the devices in the collapsed state. Many are too large in diameter, or too rigid, for navigating through small diameter arteries and through partially occluded vessels. As a consequence, most devices realistically cannot be used for carotid artery treatments or in the cerebral vasculature. Nor can the basic components of the prior art devices be scaled down in size for use in smaller arteries, due to the required cross-section of the components necessary to expand and collapse a filter-type structure.

FIGS. 1A-1B show a prior art distal protection device that may be the smallest diameter system that is commercially available and of the type disclosed in U.S. Pat. No. 6,179,861 (believed to be available in Europe; awaiting FDA approval). The system comprises a catheter housing, a nitinol expandable hoop and a basket of perforated material. In terms of the necessary functionality, (i) the perforated basket material is adapted for capturing embolic particles while allowing blood perfusion; (ii) the shape memory nitinol hoop performs the function of moving the proximal end of the basket to an expanded shape after being slidably deployed outwardly from the catheter housing; and (iii) the catheter housing is adapted for retaining the springable basket in the contracted position for navigating through and occlusion and then for returning the basket to the contracted position by retraction of the basket into the catheter bore. The entire device also may be adapted for deployment over a guidewire, which would further expand its cross section.

FIG. 1A depicts the prior art catheter being advanced through an occluded portion of an artery. FIG. 1B shows a realistic cross-section of the prior art catheter of FIG. 1B, in which the overall diameter is about 3.9 French (about 0.052″). For example, the guidewire portion 2 is about 0.14″ with each leg portion 3a and 3b of the hoop having a similar diameter. The thickness of wall 4 of the catheter housing is from about 0.005″ to 0.010″ with the thin film of the basket being foldable to fit with the catheter bore. Thus, it can be seen that the minimum cross-section C is an aggregation of the component dimensions—with no component scalable to a smaller dimension to provide a smaller cross-section C. The guidewire portion 2 is somewhat standardized in diameter for flexibility and pushability; the legs 3a and 3b of the hoop need sufficient springing strength to press against the vessel wall.

With reference to FIG. 1A, it can easily be understood that a 3.9 French catheter can be too large to navigate through many occluded vessels that are targeted for treatment.

### BRIEF DESCRIPTION OF THE DRAWINGS

FIG. 1A is a longitudinal sectional view of a blood vessel that illustrates a prior art distal protection catheter system in its contracted state being navigated through an occlusion in the blood vessel.

FIG. 1B is (i) a transverse sectional view of the prior art distal protection catheter of FIG. 1A in its contracted state showing the minimal cross-sectional dimensions of this type of system, together with (ii) a transverse sectional profile of system of the present invention in the same scale illustrating reduction in scale offered by the system of the invention.

FIG. 2 is a perspective view of a Type “A” guidewire and distal protection system corresponding to the invention in its contracted (tensioned) state.

FIG. 3 is another perspective view of the guidewire of FIG. 2 with the shape memory distal protection structure in its deployed or expanded (untensioned) state.

FIG. 4 is an enlarged view of a portion of the shape memory elements of FIG. 3 without the sac in a contracted position to show the sacrificial coupling and the insulative coating of the working end.

FIG. 5 depicts the guidewire and expanded emboli-capturing sac of FIG. 3 being collapsed and retracted into a catheter sheath for removal from the deployment site.

FIG. 6 is an alternative embodiment of a guidewire and distal protection system corresponding to the invention in its contracted state with the emboli collection sac in a cut-away view.

FIG. 7 is another view of the guidewire of FIG. 6 with the distal protection structure in its deployed or expanded state.

FIG. 8 is an alternative embodiment of a guidewire with and shape memory expandable emboli-capturing sac in its deployed state after sacrifice of a weld that maintained the sac in a collapsed position.

FIG. 9 is an another embodiment of a guidewire with a shape memory expandable emboli-capturing sac in its deployed state after sacrifice of a weld that maintained the sac in a collapsed position.

FIG. 10 is an alternative embodiment of a guidewire and distal protection system corresponding to the invention in its contracted state that utilizes a constraining sheath with an electrolytic sacrificial joint to constrain and release a shape memory structure that open a sac.

FIG. 11 is another view of the guidewire of FIG. 10 with the distal protection structure in its deployed or expanded state.

FIG. 12 is a perspective view of a Type “B” guidewire and distal protection system in its contracted state.

FIG. 13 is a perspective view of the guidewire and distal protection sac of FIG. 13 in its expanded or deployed state.

FIGS. 14A-14B are views of the support portions of the distal protection sac of FIGS. 12 & 13 in the non-extended an extended states.

FIG. 15 depicts the guidewire and expanded emboli-capturing sac of FIG. 13 being collapsed and retracted into a catheter sheath for removal from the deployment site.

FIG. 16 is a perspective view of an alternative guidewire and filter structure with support members having two free ends that expand the filter by stiffening wall portions of the filter.

FIGS. 17A-17C are enlarged views of a thin film wall of an unfolded emboli-capturing sac with an electrically responsive hydrogel layer that allow intra-operative change of the pore size of the filter wall.

FIG. 18 is a view of the strut portion of a working end of a guidewire filter structure with the strut and guidewire of a nitinol or piezoelectric element that can change its cross-section in response to electrical energy delivery thereto.

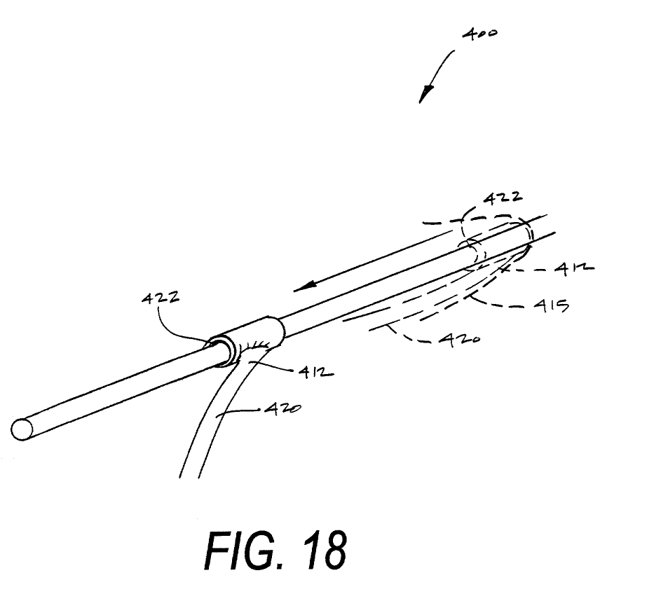

### DETAILED DESCRIPTION OF THE INVENTION

1. Type “A” Embodiment of Guidewire with Emboli Capturing Sac.

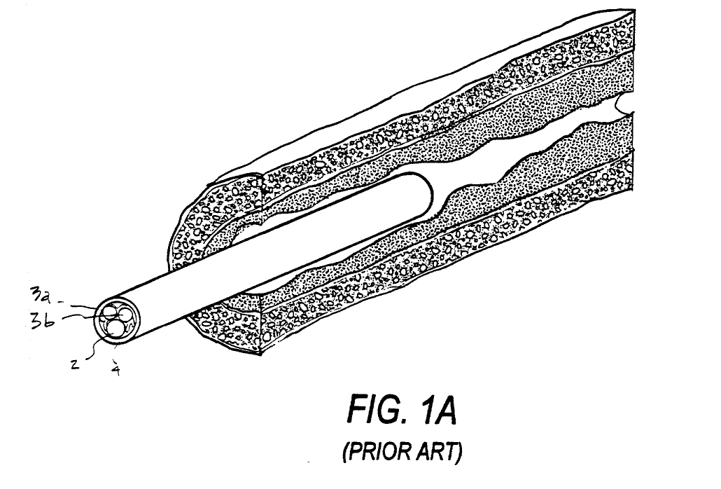

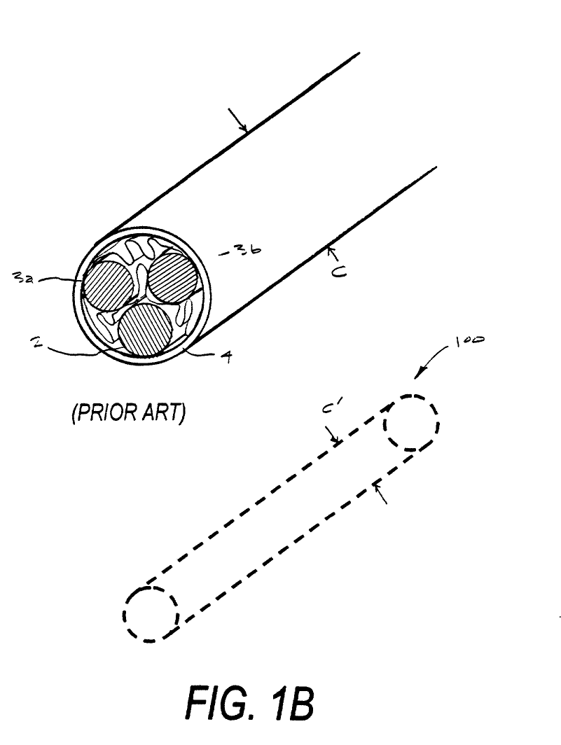

The objective of the present invention is to greatly reduce the scale of a distal protection system, in its contracted position, for navigating through tortuous arteries, severely occluded arteries and middle cerebral vasculature. As shown in FIG. 1A, it is an objective of the invention to reduce the scale of the inventive system 10 (profile in phantom view) to provide a contracted cross-section indicated at C′ that is about 40% to 50% of the diameter of the prior art system—i.e., the inventive system being about 1.5 Fr. to 2.0 Fr (about 0.020″-0.025″).

Referring again to FIG. 1B, it can be understood that a distal protection system in its contracted position can only be reduced in profile by altering the nature of the components. The present invention, in some embodiments, (i) entirely eliminates the use of a straight guidewire shaft for carrying the functional components of the filter sac and it support structure; and (ii) in all embodiments eliminates the use of a catheter sheath for constraining the springable structure in its contracted position for navigation through an occluded region of a vessel.

In other words, the present invention in some cases can be reduced in dimension to the approximate effective diameter of the guidewire and the thin film material that makes up the microporous structure for capturing emboli. Still the inventive system provides expansion means for expanding the emboli capturing structure from a contracted position to an expanded position and for springably pressing an engagement support portion of the structure into contact with the vessel wall—in manner that is improved over the prior art. Further, the structure of the emboli-capturing sac and its attachment to the guidewire allows the guidewire to have the flexibility and pushability of an unencumbered guidewire. Thus, the guidewire with embolic removal system of the present in invention can be standardized for use in practically any interventional procedure.

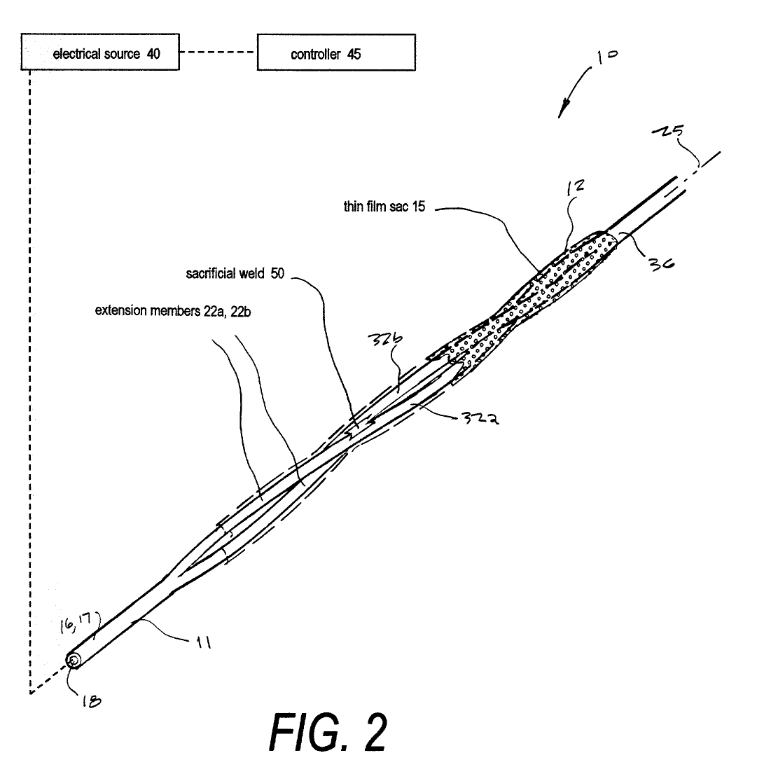

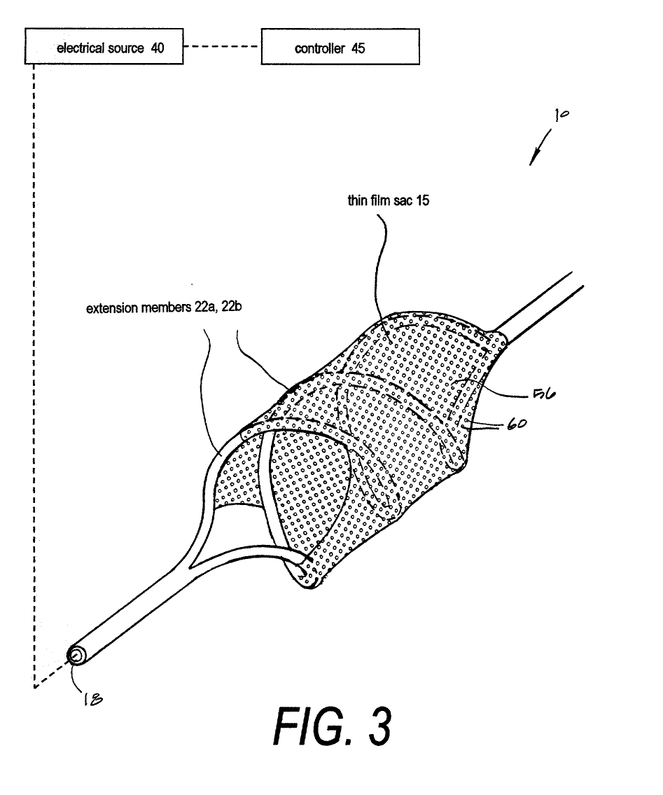

Now referring to FIG. 2, an exemplary Type “A” system 10 of the present invention is shown with of guidewire 11 having a working end 12 that carries an emboli collection structure or sac 15 in cut-away view in a first contracted state about the guidewire. FIG. 3 is a similar view of the working end 12, this time with collection sac 15 in a second extended state with the proximal sac end substantially open to allow blood flow and emboli to enter therein. As shown in FIG. 2, the guidewire 12 has proximal and medial portions 16 and 17 made of a solid metal wire core 18 without a lumen as is known in the art, and is typically about 0.014″ in diameter although any smaller or larger dimension falls within the scope of the invention. Any tapered or coiled distal tip 19 is possible (not shown) as is known in the art. FIG. 3 shows that the distal portion 20 of the guidewire comprises a plurality of shape memory extension elements 22a-22b (numbering from about two to six, with two such elements in FIG. 3) that extend generally along or about the axis 25 of the guidewire when in the first contracted state (FIG. 2).

The extension elements 22a-22b are of a type of nitinol, or nickel titanium alloy, that is known in the art as well suited for shape memory applications. Thus, FIG. 2 shows elements 22a-22b in a contracted (tensioned) state and FIG. 3 shows elements 22a-22b in an expanded (untensioned) state. In FIGS. 3 & 4, it can be seen that the core 18 of the guidewire transitions into the cores 28a and 28b of proximal ends 30a and 30b of elements 22a-22b. The extension elements 22a-22b further define medial portions 32a-32b that extend to distal ends 33a-33b that transition back into a single guidewire member portion indicated at 36. In this embodiment, the elements 22a-22b wrap around each other in a helical manner in from about one to six revolutions. FIGS. 3 & 4 further show that a thin insulative coating layer 30 covers the core 18 of the guidewire and cores 28a and 28b of elements 22a-22b. The metallic cores 18 and 28a-28b are electrically conductive and are coupled to a remote electrical source 40 and controller 45 for delivering electric current to the working end as will be described further below.

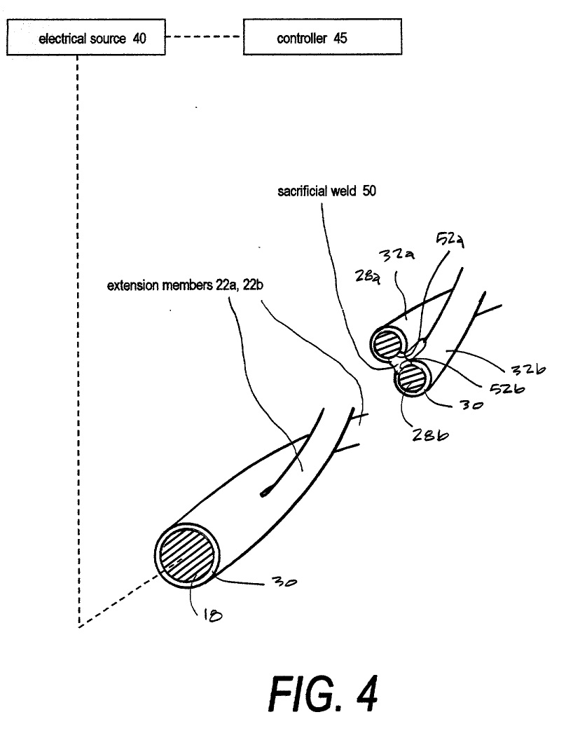

Of particular interest, FIGS. 2 and 4 further show that the two elements 22a-22b provide a constraining structure to maintained the working end and sac 15 in the first contracted and tensioned position by at least one sacrificial coupling indicated at 50. The sacrificial coupling 50 acts as a weld to bond the medial portions 32a and 32b of the elements 22a-22b together to provide the contracted profile. FIG. 2 shows a single sacrificial coupling 50 but it should be appreciated that a plurality of such discrete couplings at spaced apart locations are possible. Alternatively, one or more elongated or continuous couplings are possible and fall within the scope of the invention. FIG. 4 shows an enlarged view of a portion of one elongate element 22a with the insulative coating layer 30 removed to expose the metallic core 28a at location 52a. The other elongate element 22a is similarly provided with an exposed core portion and it is at these locations that the weld-type sacrificial coupling 50, for example of stainless steel, is provided.

As can be seen in FIGS. 2 and 3, the emboli collection sac 15 has a wall 56 of a microporous thin film polymer material known in the art with pores or perforations 60 preferably ranging between about 5 microns and 200 microns. More preferably, the pores 60 range between about 40 microns and 120 microns in dimension across a principal axis. Such microporous polymer materials are known in the art of endovascular filters, but it should be appreciated that the sac wall 56 can be any type of mesh, net, web or the like with similar dimension pores or opening therethrough. Referring to FIG. 3, the emboli collection structure 15 has a proximal portion indicated at 62a, a medial portion 62b and a distal end 62c with a proximal-facing opening portion 65 for receiving blood flow that may contain emboli. The thin film material of the sac 15 can be folded and pleated to be maintained between the elements 22a and 22b to provide the contracted position of FIG. 2.

As can be seen in FIG. 3, the emboli sac wall 56 is maintained in an expanded form by support from the elements 22a and 22b when allowed to expand to their untensioned shape. The outer portions of elements 22a and 22b are thus adapted to press against the interior of the walls of a blood vessel to insure that substantially all blood flow passes through the filter sac 15.

In use, the guidewire 10 is introduced endovascularly as is known in interventional cardiology. After the distal end 12 of the guidewire is passed beyond a stenosis or other targeted treatment site, the guidewire is maintained in a stationary position and low level direct electric current is delivered from electrical source 40 through wire core 18 to the sacrificial coupling or couplings 50. A return electrode is coupled to the patient's body by a pad or needle at a remote location to allow current flow through conductive blood to thereby cause electrolysis at the coupling 50. This system can cause electrolysis of coupling 50 until the joint fails and allows the elements 22a and 22b to spring apart to the untensioned position as depicted in FIG. 3. The delivery of an electric current to a joint is known in the detachment of an embolic coil from the distal end of a catheter in treating an intracranial aneurysm, in which the objective is the detachment of two static members. The author believes this invention is the first use of a sacrificial joint to release pent-up forces stored in a tensioned nitinol assembly or structure. The prior use of the electrolytic detachment system for embolic coils is disclosed in U.S. Pat. No. 5,855,578 and 5,122,136, incorporated herein by reference, among others authored by Guglielmi.

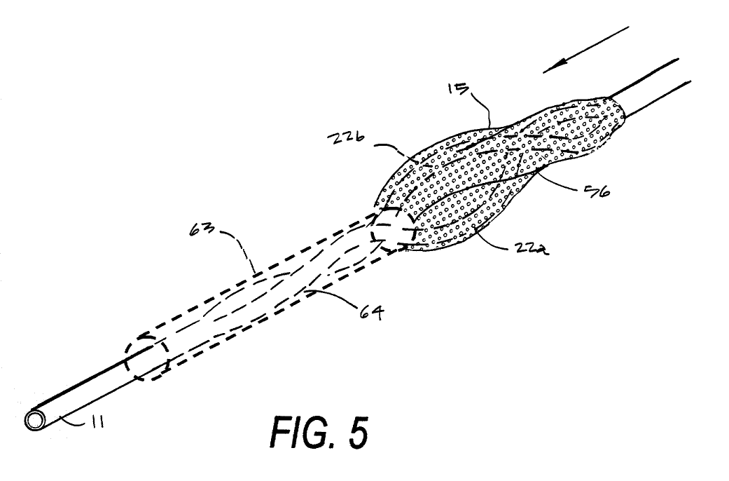

After use as an endovascular filter while performing a procedure at an upstream site (e.g., angioplastly, stent deployment, atherectomy, etc.), the guidewire 10 and sac 15 are removed from the site by advancing a catheter sleeve 63 toward the filter sac 15 and retracting the filter sac and collected emboli into a receiving bore 64 of the catheter sleeve as depicted in FIG. 5. The receiving bore 64 bore is dimensioned to collapse and receive the nitinol extension elements 22a-22b and the filter sac 15.

While FIGS. 2-4 depict two extension elements 22a and 22b that extend helically relative to one another to provide a generally round cross-section to better engage the vessel wall, it should be appreciated a working end with from 3 to 6 linear extension members of nickel titanium alloy (not shown) also can be used to extend and open a sac 15 with the medial portions of the linear elements secured in the contracted position by a electrolytically sacrificial weld.

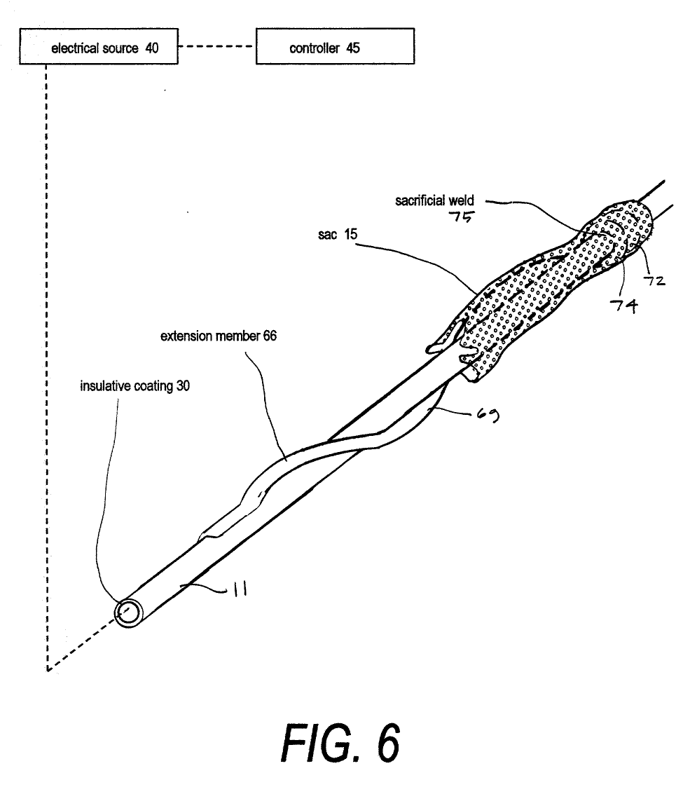

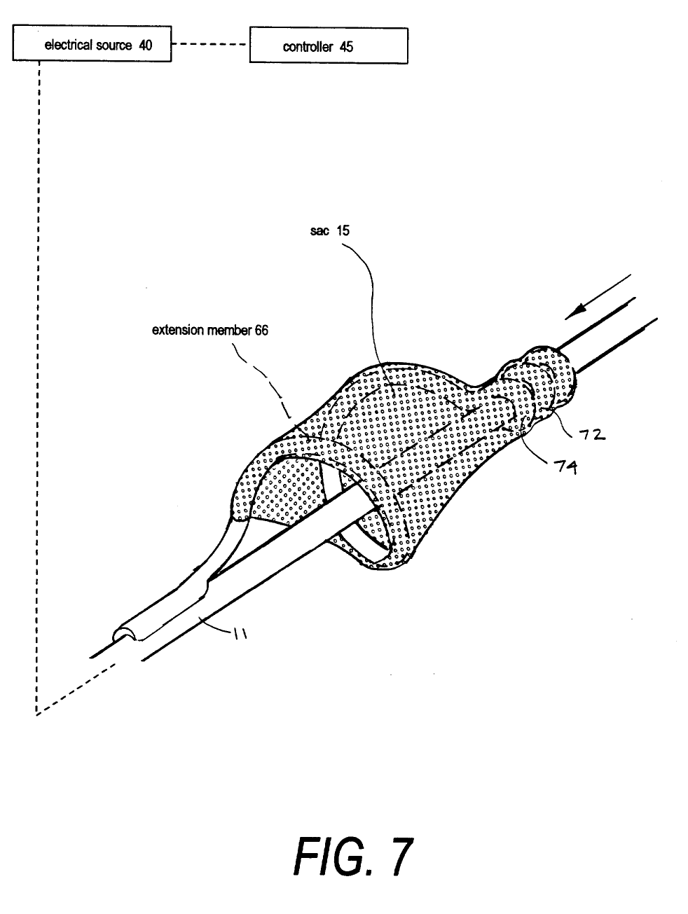

FIGS. 6 and 7 show an alternative working end 12 that is based on the principle of a sacrificial weld that can be removed by electrolysis to move a sac or basket 15 to an open position (FIG. 7) from a closed position (FIG. 6). In this embodiment, a single shape memory extension element 66 has its proximal end 68 fixedly coupled to straight guidewire 10. The medial portion 69 of the extension element 66 is helically wrapped about a straight wire portion 70 of the guidewire that is of non-shape memory material. The distal portion 72 of extension element 66 terminates in a substantially tight coil (or an optional sleeve member) that forms a sleeve portion 74 that can slide over the wire portion 70 when not welded. Thus, it can be understood that the extension member 66 can have a repose (untensioned) shape as in FIG. 7 wherein the sleeve portion 74 is slid proximally over wire portion 70. To provide a contracted position, the sleeve portion 74 can be slid distally over wire portion 70 to a tensioned state and thereafter a sacrificial weld 75 can be provided to maintain the extension member and guidewire in the low profile state. The system would be used as described previously and collapsibly retracted into a catheter sleeve following its deployment and use.

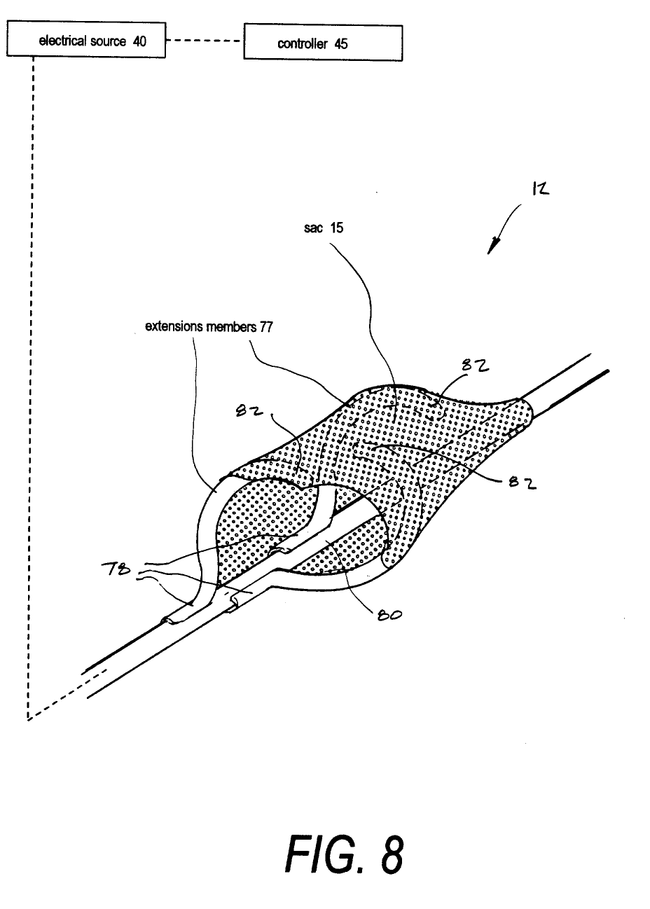

FIG. 8 shows an alternative embodiment of working end 12 based on the principle of utilizing a sacrificial weld that can be eliminated by electrolysis to open a sac 15 to the open position of FIG. 8 from a closed position (not shown). In this embodiment, the constraining structure comprises a plurality of shape memory (nitinol) extension elements 77 (collectively) have proximal ends 78 (collectively) that are fixedly coupled to the straight guidewire 10. The medial portion 79 of each extension element 77 is either linear or helically positioned against the straight portion 80 of the more rigid guidewire. To function as a constraining structure, the distal end portion 82 of each extension element 77 terminates in a free end that has a releasable weld connection (not shown) between each end 82 and the straight portion 80 of guidewire 10. After an electrolytic release, the extension elements 77 function as supports for the wall of the filter sac 15 and return to an untensioned shape that comprises a segment of an arc or hoop to open the sac. The outer surfaces 87 of the extension elements 77 are bonded to the walls of the sac to maintain the sac in a selected open configuration. It should be appreciated that the number of extension elements 77 can number from about two to eight and be coupled to the guidewire 10 at spaced apart locations or one or more proximal ends 78 of the elements 77 can be attached at single location. It is believed that this type of support members can suitably press against the vessel walls in a wider range of lumen diameters. The working end would be collapsibly retracted into a catheter sleeve following its deployment and collection of emboli.

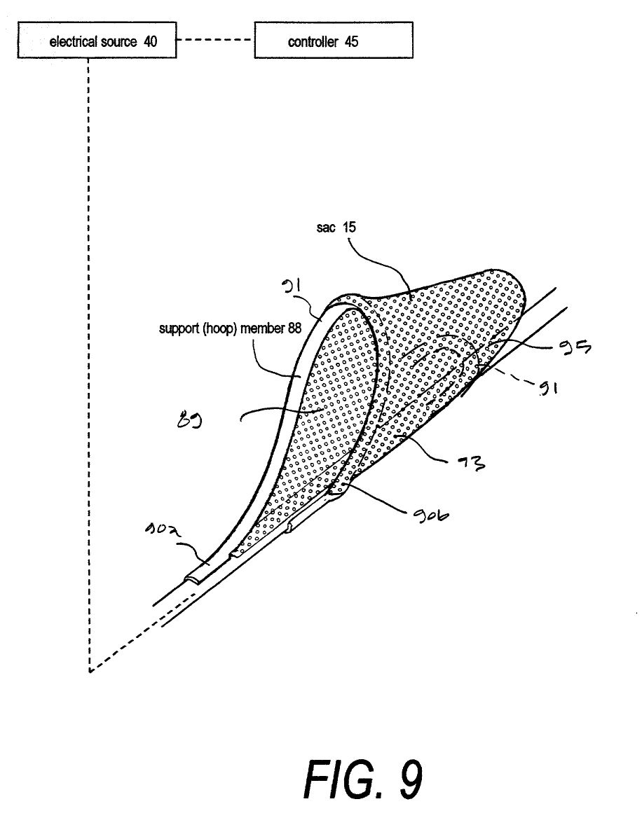

FIG. 9 shows a variation of the previous type of working end 12 based on the same principles that utilized a sacrificial weld to provide to a contracted sac position (cf. FIGS. 2 and 6) and an expanded sac position (FIG. 9). In this embodiment, at least one of shape memory (nitinol) hoop-type support member 88 is provided to provide an open mouth 89 to sac 15. The hoop member defines first and second ends 90a and 90b that are fixedly coupled to the straight guidewire 10 by a permanent weld or other bond. The medial portion 91 of the hoop element 88 is folded in the contracted position (not shown) and one or more locations 92 of the medial portion 91 of the hoop are coupled to the straight portion 93 of the guidewire (of non-shape memory material) with the sacrificial weld connection, for example at location 95 when the hoop is collapsed against the guidewire phantom view). After release delivery of electric current to cause electrolysis of the weld, the hoop-type extension element 88 will open the sac 15 as the hoop returns to the untensioned shape of FIG. 9. Again, the edges of the sac 15 are bonded to the hoop element 88 and the guidewire to maintain the sac in the open shape as the hoop is pressed against the vessel walls. The first and second ends 90a and 90b of the hoop element 88 can be coupled to the guidewire at slightly spaced apart locations as depicted in FIG. 9, or at a single location. The working end would be collapsibly retracted into a catheter sleeve following its deployment and collection of emboli as generally illustrated in FIG. 5.

The sac of FIG. 9 has its edges bonded to the hoop element 88 and to the guidewire and thus can be preformed to a desired sac shape that will deploy on one side of the guidewire. It should be appreciated that the sac of any of the above embodiments can (i) deploy on the side of the guidewire, or (ii) deploy about the guidewire with the guide wire extending through the distal end of the sac where the sac is bonded to the guidewire.

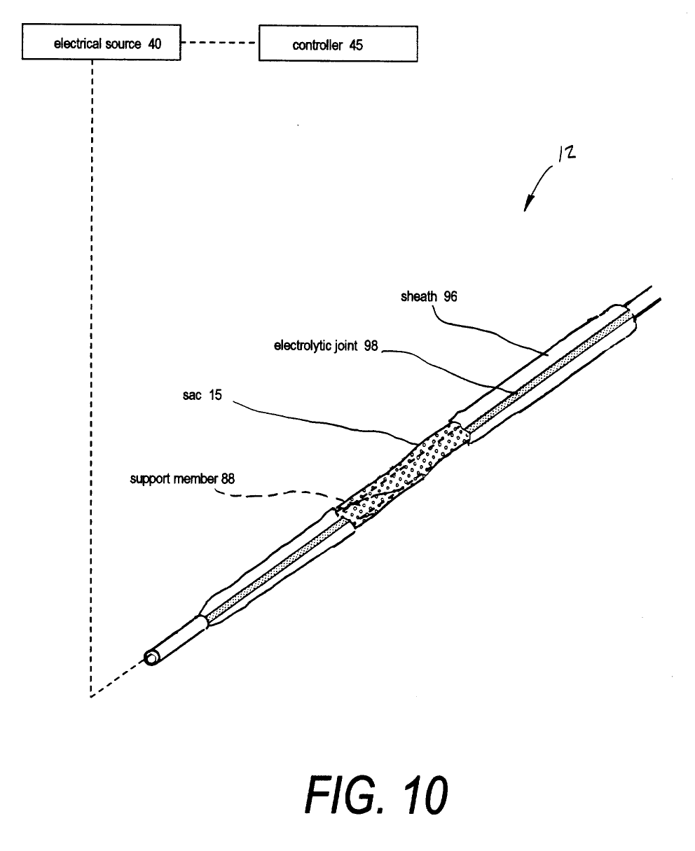

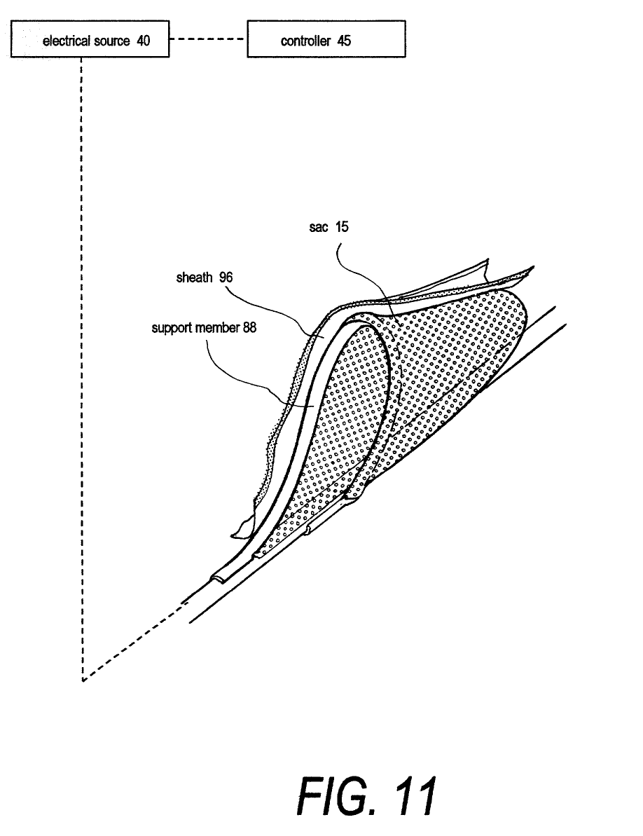

FIGS. 10 & 11 show another variation of a working end 12 that utilizes and electrical source 40 and an electrolytic sacrificial joint to release a shape memory nitinol frame or support structure that opens a emboli-collection sac 15. In this embodiment, the nitinol structure preferably is of the type shown in FIGS. 8-9, but alternatively can be any of the types described above. FIG. 10 shows the working end in a collapsed position with this embodiment providing a constraining sheath structure 96 (cut-away view) of a thin film material bonded to sac 15 along line 97 (FIG. 11). The constraining sheath structure 96 encases and retains the combination of the sac 15 and the tensioned nitinol extension elements 99 that support the sac in a contracted, tensioned position (FIG. 10). It should be appreciated that the retaining sheath structure can simply comprise a folded over portion of the sac itself. The sheath 96 in the closed position has an elongate metallic sacrificial joint 98 that comprises a thin metallic coating either or both sides of, or impregnated into, the polymer of the thin film sheath material. Upon delivery of electric current to the sacrificial joint or coupling 98 in the manner described previously, the sheath will decouple or split along the joint 98 thereby releasing the tensioned nitinol extension element(s) 99 to pop open to the untensioned position to open the emboli-capturing sac (FIG. 11). The sacrificial coupling region may have a plurality of perforations along the joint 98 to pre-weaken the targeted line of separation in the thin film polymer. The sacrificial coupling 98 is coupled to the core of the insulated guidewire as described previously to connect to the remote electrical source and controller.

2. Type “B” Embodiment of Guidewire with Emboli-Capturing Sac.

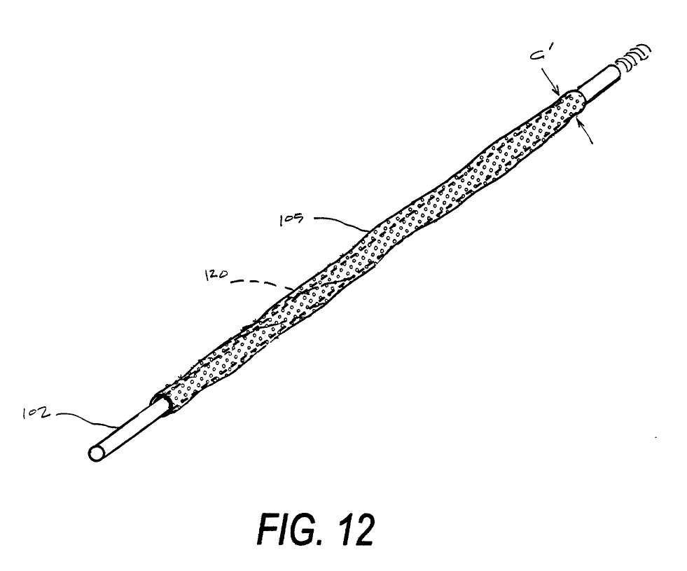

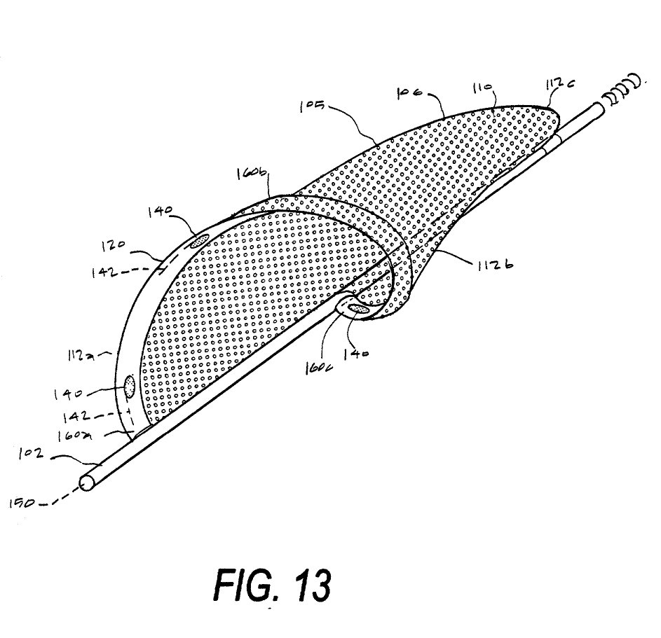

Now referring to FIG. 12, an exemplary Type “B” system 100 of the present invention is shown with the working end of guidewire 102 carrying emboli collection structure or sac 105 in a first contracted state about the guidewire shaft FIG. 13 is a similar view of the system working end, this time in a second expanded or extended state. As shown in FIG. 12, the guidewire 102 is a solid metal wire without a lumen, and can typically be about 0.014″ in diameter although any other size falls within the scope of the invention. Any tapered or coiled distal tip is possible (not shown) as is known in the art.

As can be seen in FIG. 13, the emboli collection sac 105 has a wall 106 of a microporous thin film material known in the art with pores or perforations 110 preferably ranging between about 5 microns and 200 microns. More preferably, the pores 110 range between about 40 microns and 120 microns in dimension across a principal axis. Such microporous material is known in the art of endovascular filters, but it should be appreciated that the sac wall 106 can be any type of mesh, net, web or the like with similar dimension pores or opening therethrough.

Still referring to FIG. 13, the emboli collection structure 105 has a proximal portion indicated at 112a medial portion 112b and distal end 112c with a proximal-facing open portion 115 from receiving blood flow that may contain emboli.

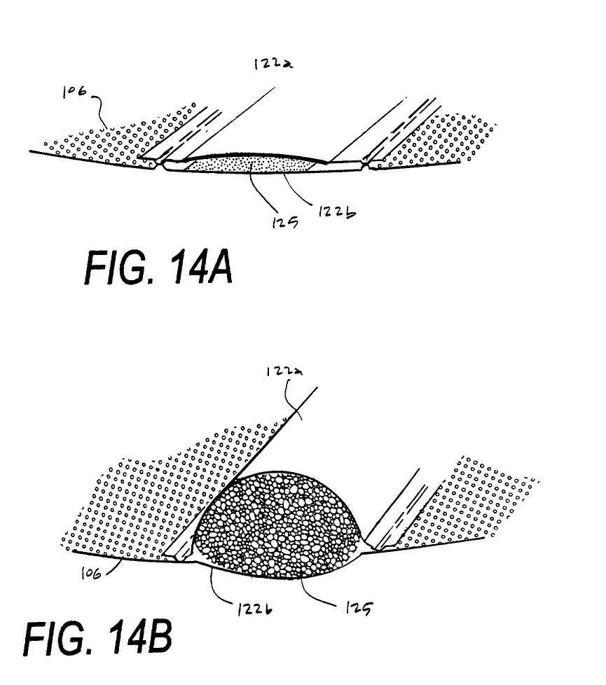

The emboli sac wall 106 is maintained in an expanded form by a support portion indicated at 120 which may also be referred to as a support member, support strut, or support rib or frame herein. Comparing FIG. 12 with FIG. 13, it can be seen that support member 120 in FIG. 12 has substantially no cross-sectional dimension wherein in FIG. 13, the support member 120 has a cross-section similar in dimension to guidewire 102. Of particular interest, to provide a support member for expanding and maintaining the emboli sac wall 106 in an expanded state, the system of the invention uses fluid from the endovascular environment—together with thin film material—to create a support member 120. More in particular, referring to FIGS. 14A-14B, the support member 120 comprises first and second film layers or sides 122a and 122b of a thin film material, e.g., two film layers with thermoseals 124a-124b, or a flattened tubular material with or without a reinforcing braid that defines sides 122a-122b. At the interior of first and second layers 122a and 122b is a volume of a desiccated porous hydrogel as in known in the art, or more preferably a desiccated microporous hydrogel indicated at 125. A microporous or superporous hydrogel is an open cell foam that can be desiccated and collapsed into a thin film or particles and disposed within the thin films layers 122a-122b. When exposed to a fluid such as blood which is substantially water, the hydrogel will expand a controlled amount to expand, stiffen and flex the support portion of or strut outwardly as in FIG. 13. The hydrogel preferably is carried within the film layers in the form of particles or strings as when the hydrogel is bonded to discrete elements of a biocompatible polymer having an suitable shape and dimension. Alternatively, the hydrogel can be coated to the film layers that contain the gel, or to other thin film elements that are tethered to the interior of the film layers 122a and 122b. The film layers 122a and 122b thus define an interior chamber indicated at 128 that contains the hydrogel and directs the swelled volume of the hydrogel to extend the containing film layer(s) in the desired direction. It is this directional extension of the film layers or tube that provides the support structure of the invention.

A suitable hydrogel can be any fast-response gel, for example of PVME, HPC or the like (see, e.g., S. H. Gehrke, Synthesis, Swelling Permeability and Applications of Responsive Gels in Responsive Gels, K. Du\check{s}ek (Ed.) Springer-Verlag (1993) pp. 86-143).

The invention further comprises a novel means or exposure mechanism for controllably exposing the hydrogel to endovascular fluids. As can be seen in FIGS. 12-13, the film layer 122a carrying the hydrogel also carries at least one sacrificial conductive film layer 140 covering a portion of chamber 128 carrying the hydrogel. Each sacrificial conductive layer portion 140 is coupled to an electrical lead 142 which in turn is coupled to conductive guidewire 102 and thereafter coupled to a remote electrical source 150. A controller 155 also is provided to control delivery energy to sacrificial layer 140 to cause electrolysis thereof to remove the layer and to thereby expose the hydrogel to blood. The system also provides a return (ground pad or needle) for coupling to the patient cause electrical potential at, or across sacrificial conductive layer 140 to cause electrolysis thereof. The sacrificial conductive film layer(s) 140 preferably are carried over porosities 156 (FIGS. 14A-14B) that have an adequate dimension to rapidly introduce fluids into chamber 128 but sufficiently small to prevent the swelled gel from escaping through the film layer.

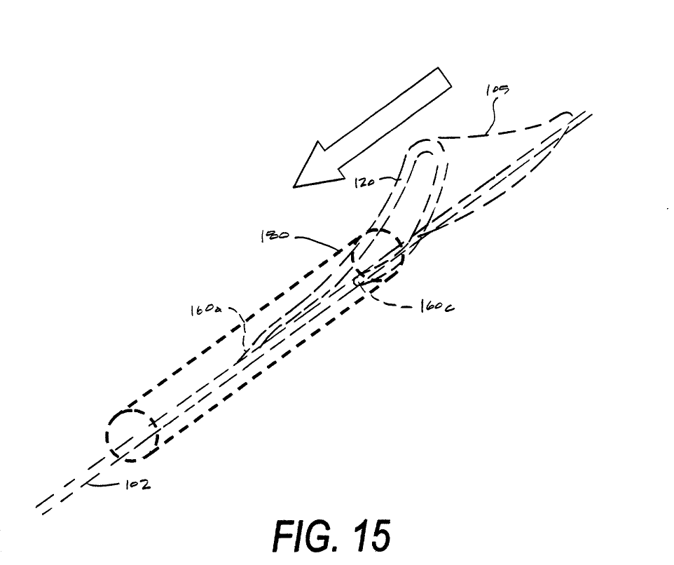

FIG. 15 depicts the Type “B” guidewire and expanded distal protection structure of FIG. 13 being collapsed and retracted into a catheter sheath 180 (phantom view) for removal from the deployment site. The method of using the system thus allows a sheath 180 of adequate size to easily receive the emboli sac which may carry a substantial amount of embolic material.

As shown in FIG. 13, the expanded support portion or strut 120 has a first end 160a, medial portion 106b and second end 160c. The first end 160a and second end 160c are can be coupled to guidewire at the same axial location, but preferable are spaced apart angularly and axially. The edge 162 of the filter film not bonded to the support member is bonded to the guidewire. Thus, a preferred embodiment has the support portion or strut 120 extending in a helical or partly helical path about the guidewire.

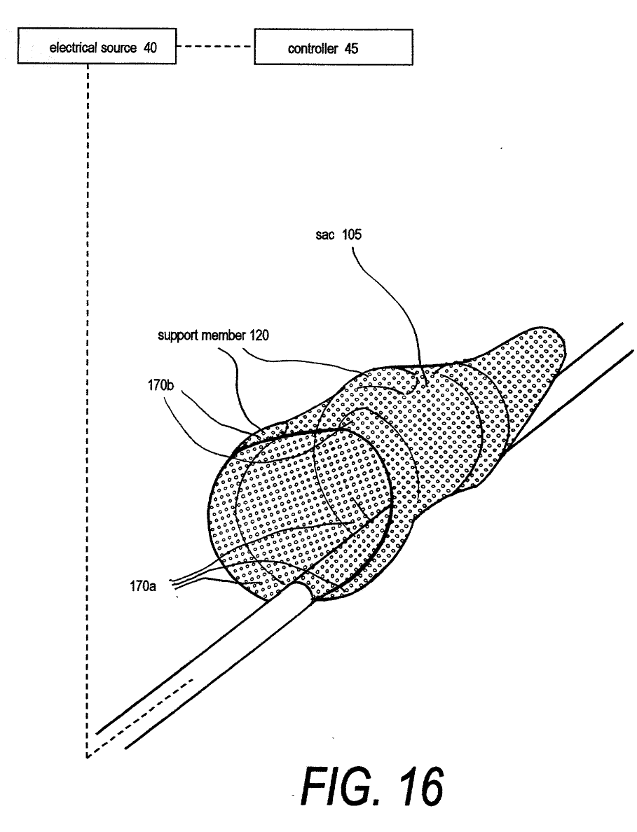

Also, a plurality of support members can be formed in a linear arrangement, instead of a helical arrangement, to open an emboli-capturing sac 105 (not shown). The emboli-containing sac 105 also can be of a thin film material wherein the proximal open end portion carries a plurality of large openings in the film wall for receiving blood flow an emboli and wherein the distal end portion of the sac has smaller filtering pores (not shown). In another embodiment, as shown in FIG. 16, the guidewire of the invention also can have an emboli-containing sac 105 that is expanded by one or more support members 120 (collectively) with one end 170a attached to the guidewire and the other free end 170b (collectively) terminating away from the guidewire but attached to the filter element 105.

It can be understood that the principles of the invention comprise (i) a support member or members 120 comprising a thin film layer around an interior chamber 128 that contains a hydrogel 125 for expanding a filter structure together with means for on-demand fluid introduction of fluids to the hydrogel from the endovascular site, and (ii) a porous filtering structure 105 coupled to the support member(s) 120 capable of a contracted or folded configuration and an expanded configuration wherein the support member(s) engage the walls of the vessel. The scope of the invention included any manner of fabricating and folding or collapsing the thin walls of support member(s) when the hydrogel is desiccated to optimize the extension of the support member(s).

3. Type “C” Embodiment of Guidewire with Emboli-Capturing Sac.

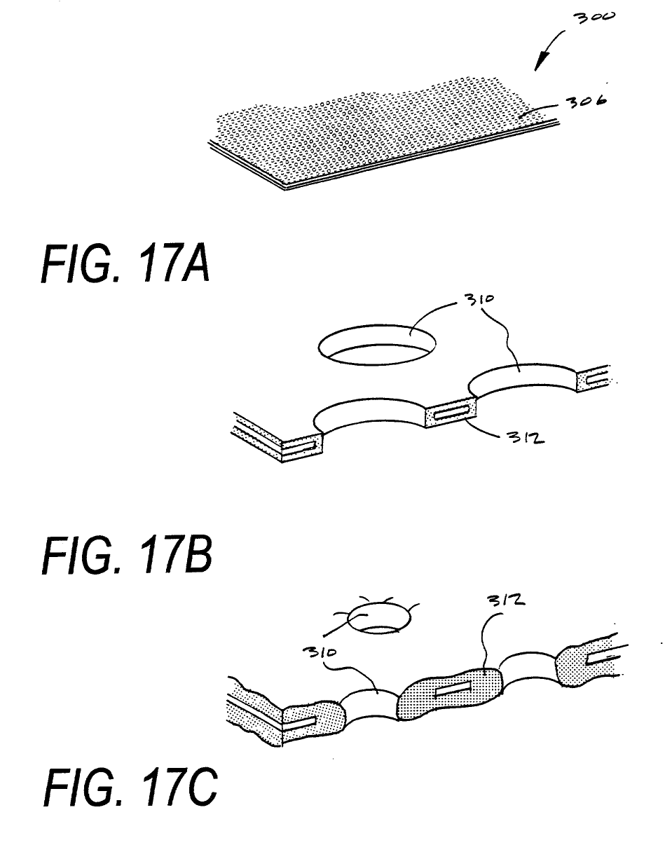

Now referring to FIG. 17A-17C, an exemplary Type “C” system 300 can be any of the above described embodiments with the improvement consisting of a new form of thin film material for the porous filter membrane of the emboli-capturing sac.

As can be seen in FIG. 24A, the emboli collection sac 305 has a wall 306 of a microporous thin film polymer material with a pores 310 therein similar to that described previously, this time for example having pores ranging between about 50 microns and 250 microns in diameter. Such porous materials are known in the art of endovascular filters, and the sac wall 306 alternatively can be any type of mesh, net, web or the like with similarly dimensioned pores or openings therein.

The improvement is depicted in the enlarged views of FIG. 17A-17C wherein the sac wall 306 carries an additional layer of a responsive hydrogel indicated at 312 which can be activated by electrical stimulation to absorb or repel water (a solute). The hydrogel extends into and about the pores 310. It can be understood that by expanding or swelling the gel, the actual pore size of the filter can be altered. By this means, it is believed that the improved emboli-capturing sac can have any variable pore dimension ranging between about 25 microns and 250 microns. This characteristic of the filter would be advantageous when deployment of the filter and imaging suggests that perfusion is higher or lower than desired—and an adjustment can be made. The hydrogel is of the type that responds to an external stimulus and preferably is an electric field responsive gel. Such gels are described in: S. H. Gehrke, Synthesis, Swelling, Permeability and Applications of Responsive Gels in Responsive Gels K. Du\check{s}ek (Ed.) Springer-Verlag (1993) pp. 86-143). Thus, the actual pore dimensions of the filter structure can be altered intra-operatively by electrical energy delivery to the hydrogel along a conductive guidewire from a remote electrical source.

In another embodiment (not shown), the interior surface of the filter sac can carry an electrolytically sacrificial layer coupled to the electrical source described above. During use, the layer could be intermittently or continuously reduces to remove platelets and other coagulative material that is smaller that embolic particles. It is believed that such a filter surface would be useful in extending the treatment time, wherein a typical filter may begin to clog due to the fibrogenic cascade that occurs about the foreign object in the vasculature.

4. Type “D” Guidewire with Emboli-Capturing Sac.

FIG. 25 depicts a Type “D” embodiment of guidewire 400 that carries expandable emboli-capturing structure 415 at it distal end. This embodiment is similar to that of FIG. 6-7 which have a sleeve portion that is detachably coupled to the guidewire in a tensioned position. The Type “D” embodiment utilizes an electrically activated release mechanism that comprises a nickel titanium sleeve or a piezoelectric sleeve that is moveable between first and second dimensions to release the distal end 412 of a tensioned support member 420 from a guidewire portion indicated at 410. The distal end 412 of a support member 420 carries the sleeve that can change the dimension of its bore 422 to compress and grip the fixed diameter guidewire. It is well known in the art that electrical energy can be delivered to a nitinol sleeve to cause resistive heating thereof to cause a change in its dimension to a remembered condition. In use, the physician extends and tensions the support member 420 to provide the contracted position and then actuates the electrical source to alter the dimension of the sleeve 420 to maintain the structure in the contracted position. After introducing the working end to the targeted location, electrical energy is delivered to the sleeve to altered its cross-section to release the coupling from the guidewire to thereby open and expand the emboli-capturing structure 415 to the second expanded position.

Those skilled in the art will appreciate that the exemplary embodiments and descriptions thereof are merely illustrative of the invention as a whole. While the principles of the invention have been made clear in the exemplary embodiments, it will be obvious to those skilled in the art that modifications of the structure, arrangement, proportions, elements, and materials may be utilized in the practice of the invention, and otherwise, which are particularly adapted to specific environments and operative requirements without departing from the principles of the invention.

What is claimed is:

1. A guidewire apparatus for endovascular interventions, comprising:
an elongate guide member extending along an axis from a proximal end to a distal working end,
a filter sac coupled to said working end;
at least one shape memory support element coupled to the filter sac, the filter sac moveable between a tensioned-collapsed position and an untensioned-expanded position; and
a releasable constraint structure for maintaining the filter sac in the tensioned-collapsed position, the releasable constraint structure comprising at least one sacrificial coupling between portions of the constraint structure.

2. The guidewire apparatus of claim 1 wherein the at least one sacrificial coupling is of an electrolytically responsive material.

3. The guidewire apparatus of claim 1 further comprising a remote electrical source coupled to the at least one sacrificial coupling.

4. The guidewire apparatus of claim 2 wherein said at least one coupling is of stainless steel.

5. The guidewire apparatus of claim 1 wherein the releasable constraint structure comprises the least one shape memory support element and the guide member with at least one sacrificial coupling therebetween.

6. The guidewire apparatus of claim 1 wherein the releasable constraint structure comprises a plurality of axially-extending shape memory support elements with at least one sacrificial coupling between portions thereof.

7. The guidewire apparatus of claim 1 wherein the releasable constraint structure comprises at least one hoop-type shape memory element and the guide member with said at least one sacrificial coupling therebetween.

8. The guidewire apparatus of claim 1 wherein the releasable constraint structure comprises a thin film member with a sacrificial coupling between portions thereof.

9. The guidewire apparatus of claim 8 wherein the thin film member comprises a sheath with a sacrificial coupling between portions thereof.

10. The guidewire apparatus of claim 1 wherein the at least one shape memory support element extends at least partially in a hoop in the untensioned-expanded position.

11. The guidewire apparatus of claim 1 wherein the at least one shape memory support element extend at least partially helically relative to said guide member in the tensioned-collapsed position.

12. A method for performing an endoluminal procedure, comprising the steps of:
(a) providing a guidewire member having a distal working end that carries an expandable-collapsible filter sac coupled to at least one shape memory support element, and a releasable constraint structure for maintaining the filter sac in the tensioned-collapsed position;
(b) advancing the working end endovascularly to a deployment site with the filter sac in the tensioned-collapsed position; and
(c) delivering electrical current to at least one electrolytic sacrificial coupling carried by the constraint structure to thereby release the filter sac to move from the tension-collapsed position to an untensioned-expanded position.

13. The method of claim 12 further including the steps of performing a medical intervention proximal to the filter sac and capturing emboli within said filter sac.

14. A method of claim 13 further including collapsibly retracting the filter sac with captured emboli therein into the bore of a catheter and retracting the assembly from the endoluminal site.

15. A guide wire apparatus for endovascular interventions, comprising:
an elongate guide wire extending along an axis to a working end; and
a filter structure coupled to said working end, the filter structure comprising a thin film filter member and a shape memory support member, the shape memory member having a first end portion fixedly coupled to said guide wire and another portion thereof releasably coupled to said guide wire with an electrically releasable coupling.

16. The guide wire apparatus of claim 15 wherein the filter structure is capable of a contracted position in which the shape memory support member is in a tensioned state extending about said axis and an expanded position in which the shape memory support member is in an untensioned state extending away from said axis.

17. The guide wire apparatus of claim 15 wherein the electrically releasable coupling is selected from the class consisting of electrolytic sacrificial couplings, nitinol couplings and piezoelectric couplings.

18. The guide wire apparatus of claim 15 wherein the guidewire has a conductive core portion and an insulative surface layer except for the region of the electrically releasable coupling.

19. The guide wire apparatus of claim 15 wherein the first and second ends of the shape memory support member are fixedly coupled to said guide wire with a medial portion thereof coupled to the guidewire with an electrolytic sacrificial coupling.

20. The guide wire of claim 15 wherein the filter sac has pores with an average dimension across a principal axis ranging between about 5 microns and 200 microns.
